(19) United States
(12) Patent Application Publication
(10) Pub. No.: US 2024/0058326 A1
(43) Pub. Date: Feb. 22, 2024

(54) METHODS OF TREATING MENTAL OR MOOD DISORDERS USING 2-BROMO-LSD

(71) Applicant: BETTERLIFE PHARMA INC., Vancouver (CA)

(72) Inventors: Justin KIRKLAND, Champaign, IL (US); Patrick KROUPA, Olivebridge, NY (US)

(21) Appl. No.: 18/269,080

(22) PCT Filed: Dec. 22, 2021
(86) PCT No.: PCT/CA2021/051869

§ 371 Date: Jun. 22, 2023

Related U.S. Application Data
(60) Provisional application No. 63/129,116, filed on Dec. 22, 2020.

(51) Int. Cl. A61K 31/48; A61K 9/48; A61K 9/20; A61K 9/00

(52) U.S. Cl. CPC A61K 31/48; A61K 9/4866; A61K 9/4858; A61K 9/485; A61K 9/2013; A61K 9/2054; A61K 9/2009; A61K 9/0056; A61K 9/2018

(57) ABSTRACT

Methods using 2-Bromo-LSD or a derivative or salt thereof to treat subjects suffering from mental and/or mood disorders. The 2-Bromo-LSD is provided as a pharmaceutical composition formulated in a variety of manners for resolving symptoms even in subjects who have failed prior therapy. Methods comprise or consist of administering 2-bromolysergic acid diethylamide or a derivative or salt thereof to a subject in need of.

### BACKGROUND

Major depressive disorder (MDD) is a psychiatric disorder which has a lifetime prevalence of around 8%. MDD is characterized by increased medical morbidity, mortality, functional impairment, reduced quality of life, substantial health-care costs, and an increased risk of suicide, loss of interest or pleasure, disturbed sleep or appetite, low energy, and feelings of guilt or low self-worth (Greden et al, J Clin Psychiat. 2001, 62: 26-31; Tranter et al, J Psychiat Neurosci. 2002, 27: 241-247; Uher et al, Depress Anxiety. 2014, 31: 459-471). MDD is one of the most common mental disorders worldwide, with a life time prevalence of 16.2% and a 12-month prevalence of 6.6% in developed countries (Trivedi et al, CNS Spectr. 2007, 12: 1-27). According to the World Health Organization (WHO, 2010), MDD carries the heaviest burden of disability among mental and behavioral disorders. The findings from the 2014 National Survey on Drug Use and Health (NSDUH) revealed that in 2014, 11.4 percent of youths aged 12 to 17 (2.8 million adolescents) had a major depressive episode (MDE) in the past year (Center for Behavioral Health Statistics and Quality, 2015). Youths aged 12 to 17 in 2014 who had a past year MDE were more likely than those without a past year MDE to have used any illicit drugs in the past year (33.0 vs. 15.2 percent). Nierenberg and DeCecco concluded that though the initial antidepressant therapy reduces symptoms of depression in patients, but only 50-60% of patients with major depressive disorder respond to therapy (Nierenberg and DeCecco, J Clin Psychiat. 2001, 62: 5-9). Furthermore, between 30 and 40% of patients who suffer from MDD never achieve symptom resolution with standard antidepressant treatment (Amsterdam and HornigRohan, Psychiatr Clin North Am. 1996, 19: 371-386; Nierenberg and Amsterdam, J Clin Psychiat. 1990, 51: 39-47).

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

References in the specification to “one embodiment.” “an embodiment,” “an illustrative embodiment.” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally. additional unlisted items. Only terms clearly indicated to the contrary, such as “only one of” or “exactly one of,” or, when used in the claims, “consisting of,” will refer to the inclusion of exactly one element of a number or list of elements. In general. the term “or” as used herein shall only be interpreted as indicating exclusive alternatives (e.g. “one or the other but not both”) when preceded by terms of exclusivity, such as “either” “one of,” “only one of” or “exactly one of.” “Consisting essentially of,” when used in the claims, shall have its ordinary meaning as used in the field of patent law.

As used herein, the terms “comprising,” “including.” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order. the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and or B”) can refer, in one embodiment, to at least one, optionally including more than one. A, with no B present (and optionally including elements other than B): in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the phrases “effective amount” or “therapeutically effective amount” (used interchangeably herein) refer to the amount of a composition or formulation described herein that will elicit the diagnostic, biological or medical response of a tissue, system, animal, or human that is being sought by the researcher, veterinarian, medical doctor or other clinician.

As used herein, the terms “pharmaceutically acceptable excipient,” “carrier,” or “diluent” refer to pharmaceutical components which do not alter the therapeutic properties of an active agent with which it is administered. One exemplary pharmaceutically acceptable carrier substance is physiological saline. For instance, the pharmaceutically acceptable carrier can include sodium chloride (e.g., 150 mM sodium chloride) and sodium phosphate (e.g., 25 mM sodium phosphate). Other physiologically acceptable excipients, carriers, and diluents, and their formulations, are known to those skilled in the art and described, e.g., in Remington: The Science and Practice of Pharmacy (22nd Ed), Allen (2012). For instance, a pharmaceutically acceptable excipient, carrier, or diluent can include dibasic sodium phosphate, heptahydrate; monobasic sodium phosphate, monohydrate; and sodium chloride at a pH of about 7.2 to 7 about 0.6.

As used herein, the term “pharmaceutical composition” it is meant a composition containing an active agent as described herein, formulated with at least one pharmaceutically acceptable excipient, carrier, or diluent. The pharmaceutical composition can be manufactured or sold with the approval of a governmental regulatory agency as part of a therapeutic regimen for the treatment or prevention of a disease or event in a patient (e.g., an infant with HPP, such as an infant having perinatal-onset HPP, or an infant having infantile-onset HPP, or juvenile-onset HPP, or a patient having childhood-onset HPP). Pharmaceutical compositions can be formulated, for example, for subcutaneous administration, intravenous administration (e.g., as a sterile solution free of particulate emboli and in a solvent system suitable for intravenous use), for oral administration (e.g., a tablet, capsule, caplet, gelcap, or syrup), or any other formulation described herein, e.g., in unit dosage form.

As used herein, the term “subject” refers to any animal subject including laboratory animals (e.g., primates, rats, mice), livestock (e.g., cows, sheep, goats, pigs, turkeys, chickens), household pets (e.g., dogs, cats, rodents, etc.), and humans. Typically, the mammal is a human (Homo sapiens).

As used herein, the terms “treatment,” “treating,” or “treat,” with respect to a specific condition (e.g. major depressive disorder), refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment”, as used herein, covers any treatment of a disease or disorder in a subject, particularly in a human, and includes: (a) preventing the disease or disorder from occurring in a subject which may be predisposed to the disease but has not yet been diagnosed as having it; (b) inhibiting the disease or disorder, i.e., arresting its development; and (c) relieving or alleviating the disease or disorder, i.e., causing regression of the disease or disorder and/or relieving one or more disease or disorder symptoms. “Treatment” can also encompass delivery of an agent or administration of a therapy in order to provide for a pharmacologic effect, even in the absence of a disease, disorder or condition. The term “treatment” is used in some embodiments to refer to administration of a compound of the present disclosure to mitigate a disease or a disorder in a host, preferably in a mammalian subject, more preferably in humans. Thus, the term “treatment” can include preventing a disorder from occurring in a host, particularly when the host is predisposed to acquiring the disease but has not yet been diagnosed with the disease; inhibiting the disorder; and/or alleviating or reversing the disorder. As far as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure can occur prior to onset of a disease. The term does not mean that the disease state must be completely avoided.

### OVERVIEW/SUMMARY

It would be desirable to treat subjects suffering from mental and/or mood disorders with an improved pharmaceutical composition that is able to resolve symptoms even in subjects who have failed prior therapy. As such, it is an aspect of the present disclosure to provide a method for treating a mental disorder and/or a mood disorder in a subject in need of treatment thereof.

Methods using 2-Bromo-LSD or a derivative or salt thereof are developed and described herein to treat subjects suffering from mental and/or mood disorders. The 2-Bromo-LSD is provided as a pharmaceutical composition formulated in a variety of manners for resolving symptoms even in subjects who have failed prior therapy. Methods comprise or consist of administering 2-bromolysergic acid diethylamide or a derivative or salt thereof to a subject in need of. The formulations described herein are self-administrable.

In some embodiments, the present disclosure relates to a method of treating or therapeutically managing a mental disorder or a mood disorder in a subject in need of treatment thereof, the method comprising administering an effective amount of 2-bromolysergic acid diethylamide (hereinafter “2-Bromo-LSD”) or a derivative or salt thereof, or a pharmaceutical composition comprising 2-Bromo-LSD or a derivative or salt thereof. 2-Bromo-LSD and its method of synthesis are described in U.S. Pat. Nos. 9,868,732 and 10,377,752, the disclosures of which are hereby incorporated by reference herein in their entireties. The chemical structure of 2-Bromo-LSD is depicted below.

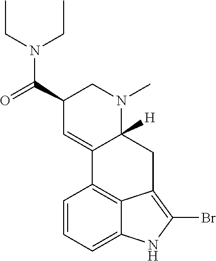 [SMILES: CCN(CC)C(=O)[C@@H]1C=C2c3cccc4[nH]c(Br)c(c34)C[C@H]2N(C)C1]

In some embodiments, the mental disorder or mood disorder is depression.

In some embodiments, the mental disorder or the mood disorder is major depressive disorder.

In other embodiments, the mental disorder or mood disorder is Attention Deficit Hyperactivity Disorder.

In other embodiments, the mental disorder or mood disorder is Obsessive-Compulsive Disorder.

In other embodiments, the mental disorder or mood disorder is Atypical Depression.

In other embodiments, the mental disorder or mood disorder is Psychotic major depression.

In other embodiments, the mental disorder or mood disorder is Catatonic Depression.

In other embodiments, the mental disorder or mood disorder is Postpartum Depression.

In other embodiments, the mental disorder or mood disorder is Premenstrual Dysphoric Disorder.

In other embodiments, the mental disorder or mood disorder is Seasonal Affective Disorder.

In other embodiments, the mental disorder or mood disorder is Dysthymia. In other embodiments, the mental disorder or mood disorder is Double Depression.

In other embodiments, the mental disorder or mood disorder is Depressive Disorder Not Otherwise Specified.

In other embodiments, the mental disorder or mood disorder is Depressive Personality Disorder.

In other embodiments, the mental disorder or mood disorder is Recurrent Brief Depression.

In other embodiments, the mental disorder or mood disorder is Minor Depressive Disorder.

In other embodiments, the mental disorder or mood disorder is a Bipolar Disorder (e.g. Bipolar I, Bipolar II, Cyclothymia, Bipolar disorder not otherwise specified, Impulse-Control Disorders, Adjustment Disorders, Personality Disorders).

An effective amount of 2-Bromo-LSD or a derivative or a salt thereof, as described herein, will provide therapeutic benefit without causing substantial toxicity. The skilled artisan will appreciate that the toxicity of 2-Bromo-LSD or a derivative or a salt thereof can be determined by standard pharmaceutical procedures in cell cultures or experimental animals, for example, by determining the LD50 (the dose lethal to 50% of the population) or the LD100 (the dose lethal to 100% of the population). In some embodiments, the dose ratio between toxic and therapeutic effect is the therapeutic index. In some embodiments, the data obtained from these cell culture assays and animal studies can be used in formulating a dosage range that is not toxic for use in human. In some embodiments. the dosage of the compounds described herein lies within a range of circulating concentrations that include the effective dose with little or no toxicity. In some embodiments, the dosage may vary within this range depending upon the dosage form employed and the route of administration utilized. In some embodiments, the exact formulation, route of administration and dosage can be chosen by the individual physician in view of the patient's condition. (See, e.g., Fingl et al., 1996, In: The Pharmacological Basis of Therapeutics, 9th ed., Chapter 2, p. 29, Elliot M. Ross)

Examples of therapeutically effective doses of 2-Bromo-LSD for various mental and/or mood disorders are set forth below. In some embodiments, the term “about” when used in reference to the amount of 2-Bromo-LSD or derivative or salt thereof means about +/−1%. In some embodiments, the term “about” when used in reference to the amount of 2-Bromo-LSD or derivative or salt thereof means about +/−2%. In some embodiments, the term “about” when used in reference to the amount of 2-Bromo-LSD or derivative or salt thereof means about +/−2.5%. In some embodiments, the term “about” when used in reference to the amount of 2-Bromo-LSD or derivative or salt thereof means about +/−5%. In some embodiments, the term “about” when used in reference to the amount of 2-Bromo-LSD or derivative or salt thereof means about +/−10%. In some embodiments, the tern “about” when used in reference to the amount of 2-Bromo-LSD or derivative or salt thereof means about +/−15%. In some embodiments, the term “about” when used in reference to the amount of 2-Bromo-LSD or derivative or salt thereof means about +/−20%.

| | μg/kg/ |
|---|---|
| Mental and/or Mood Disorder | bodyweight/day |
| Major Depressive Disorder | about 30 |
| Attention Deficit Hyperactivity Disorder | about 15 |
| Obsessive Compulsive Disorder | about 30 |
| Atypical Depression | about 30 |
| Melancholid Depression | about 30 |
| Psychotic major depression | about 30 |
| Catatonic Depression | abous 45 |
| Postpartum Depression | about 30 |
| Premenstrual dysphoric disorder | about 25 |
| Seasonal affective disorder | about 25 |
| Dysthymia | about 30 |
| Double Depression | about 30 |
| Depressive Disorder Not Otherwise Specified | about 30 |
| Depressive personality disorder | about 30 |
| Recurrent brief depression | about 30 |
| Minor Depressive disorder | about 15 |
| Bipolar Disorders: | about 30 |
| Bipolar I | about 30 |
| Bipolar II | about 30 |
| Cyclothymia | about 30 |
| Bipolar disorder not otherwise specified | about 30 |
| Impulse-Control Disorders | about 30 |
| Adjustment Disorders | about 30 |
| Personality Disorders | about 45 |

### Pharmaceutically Acceptable Carriers

Pharmaceutically acceptable carriers include diluents and excipients generally used in pharmaceutical preparations, such as fillers, extenders, binders, moisturizers, disintegrators, surfactant, lubricants, etc. Non-limiting examples of suitable carriers are described herein.

### Diluents

A diluent may be selected from, for example, calcium carbonate, calcium phosphate dibasic, calcium phosphate tribasic, calcium sulfate, microcrystalline cellulose, microcrystalline silicified cellulose, powdered cellulose, dextrate, dextrose, fructose, lactitol, lactose anhydrous, lactose monohydrate, lactose dihydrate, lactose trihydrate, mannitol, sorbitol, starch, pregelatinized starch, sucrose, tale, xylitol, maltose, maltodextrin, maltitol. In some embodiments, the diluent is selected from starches, lactose, cellulose derivatives, confectioner's sugar and the like. Different grades of lactose include, but are not limited to, lactose monohydrate, lactose DT (direct tableting), lactose anhydrous, and others. Different starches include, but are not limited to, maize starch, potato starch, rice starch, wheat starch, pregelatinized starch, and others. Different celluloses that can be used include crystalline celluloses, such as a microcrystalline cellulose, and powdered celluloses. Other useful diluents include, but are not limited to, carmellose, sugar alcohols such as mannitol, sorbitol, and xylitol, calcium carbonate, magnesium carbonate, dibasic calcium phosphate, and tribasic calcium phosphate.

### Binders

A binder may be selected from, for example, acacia, alginic acid, carbomer. carboxymethylcellulose calcium, carbomethylcellulose sodium, microcrystalline cellulose, powdered cellulose, ethyl cellulose, gelatin liquid glucose, guar gum, hydroxyethyl cellulose, hydroxypropyl cellulose, hydroxypropylmethyl cellulose, maltodextrin, methylcellulose, polydextrose, polyethtylene oxide, povidone, sodium alginate, starch paste, pregelatinized starch, sucrose, tragacanth, low-substituted hydroxypropyl cellulose, glucose, sorbitol.

### Fillers

A suitable filler may be selected from, for example, starch derivatives, such as com starch, potato starch or rice starch, polysaccharides such as dextrins, maltodextrins, dextrates, microcrystalline cellulose, powdered cellulose, mixture of microcrystalline cellulose and guar gum, coprocessed blends of microcrystalline cellulose; and polyhydric alcohols, such as xylitol and sorbitol.

### Disintegrants

A disintegrant may be selected from, for example, alginic acid, carbon dioxide, carboxymethylcellulose calcium, carboxymethylcellulose sodium, microcrystalline cellulose, powdered cellulose, croscarmelose sodium, crospovidone, sodium docusate. gaur gum, hydroxypropyl cellulose, methylcellulose, polacrilin potassium, poloxamer, povidone, sodium alginate, sodium glycine carbonate, sodium lauryl sulfate, sodium starch glycolate, starch, pregelatinized starch, low-substituted hydroxypropyl cellulose.

### Glidants

A glidant may be selected from, for example, calcium silicate, powdered cellulose, starch, talc, colloidal silicon dioxide.

### Lubricants

A lubricant may be selected from, for example, magnesium stearate, stearic acid, sodium stearyl fumarate, magnesium lauryl sulphate, talc, polyethylene glycol, and glyceryl behenate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and any combinations thereof.

The pharmaceutical composition of the present disclosure may be formulated as an ordinary pharmaceutical preparation, for example in the form of tablets, flash melt tablets, pills, powder, liquid, suspension, emulsion, granules, capsules, suppositories or injection (liquid, suspension, etc.), troches, intranasal spray percutaneous patch and the like.

### Absorption Enhancers

Absorption enhancers for use in accordance with certain embodiments of the present disclosure include, for example, Gelucire 44/14; Gelucire 50/13; Tagat TO; Tween 80; isopropyl myristate, polysorbates, sorbitan esters, poloxamer block copolymers, PEG-35 castor oil, PEG-40 hydrogenated castor oil, caprylocaproyl macrogol-8 glycerides, PEG-8 caprylic/capric glycerides, sodium lauryl sulfate, dioctyl sulfosuccinate, polyethylene lauryl ether, ethoxydiglycol, propylene glycol mono-di-caprylate, glycerol monocaprylate, glyceryl fatty acids (C8-C18) ethoxylated, oleic acid, linoleic acid, glyceryl caprylate caprate, glyceryl monooleate, glyceryl monolaurate, caprylic/capric triglycerides, ethoxylated nonylphenols, PEG-(8-50) stearates, olive oil PEG-6 esters, triolein PEG-6 esters, lecithin, d-alpha tocopheryl polyethylene glycol 1000 succinate, polycarbonate, sodium glycocholate, sodium taurocholate. cyclodextrins, citric acid, sodium citrate, triacetin, combinations thereof, and the like. In certain preferred embodiments, the absorption enhancer is triacetin.

### Sweeteners/Flavoring Agents

A suitable sweetener may be selected from sugars such as sucrose, lactose and glucose; cyclamate and salts thereof; saccharin and salts thereof; and aspartame. Flavoring agents may be incorporated in the composition may be chosen from synthetic flavors oils and flavoring aromatics, natural oils, plant extracts. Examples include cinnamon oil, oil of wintergreen, peppermint oil, clove oil, bay oil, anise oil, eucalyptus, thyme oil, cedar leaf oil, nutmeg oil, sage oil or almond oil. Examples of flavoring agents include, but are not limited to, almond, apple, banana, berry, bubblegum, caramel, citrus, cherry, chocolate, coconut, grape, green tea, honey, lemon. licorice, lime, mango, maple, mint, orange, peach, pineapple, raisin, strawberry, vanilla, watermelon and combinations thereof. Flavors may be present in an amount ranging from about 0.001001% to about 5% by total weight of the formulation. In some embodiments, the flavoring agent may be selected from natural or synthetic flavors such as, for example, strawberry flavor, wild cherry flavor, green apple flavor, spearmint flavor and peppermint flavor. In some embodiments, the flavoring agents are selected from menthol, peppermint, wintergreen, orange, cherry, and other fruits, vanilla, almond and other nuts, etc.

In some embodiments the pharmaceutical compositions of the present disclosure are in the form of tablets, which may include one or more pharmaceutically acceptable carriers or excipients selected from lactose, saccharose, sodium chloride, glucose, urea. starch, xylitol, mannitol, erythritol, sorbitol, calcium carbonate, kaolin, crystalline cellulose, silic acid and other excipients; water, ethanol, propanol, simple syrup, glucose solution, starch solution, gelatin solution, carboxymethyl cellulose, shellac, methyl cellulose, potassium phosphate, polyvinyl pyrrolidone and other binders; dried starch, sodium alginate, agar powder, laminaran powder, sodium hydrogencarbonate, calcium carbonate, polyoxyethylene sorbitan fatty acid esters, sodium lauryl sulfate, stearic acid monoglyceride, starch, lactose and other disintegrators; white sugar, stearin, cacao butter, hydrogenated oil and other disintegration inhibitors; quaternary ammonium salt, sodium lauryl sulfate and other absorption accelerator; glycerine, starch and other moisture retainers; starch, lactose, kaolin, bentonite, colloidal silic acid and other adsorbents; and refined talc, stearate, boric acid powder, polyethylene glycol and other lubricants and the like. Tablets can also be formulated with ordinary coatings, such as sugar-coated tablets, gelatin-coated tablets, enteric coated tablets and film coated tablets, as well as double tablets and multilayered tablets.

In some embodiments the pharmaceutical compositions of the present disclosure are in the form of pills, which may include one or more pharmaceutically acceptable carriers or excipients selected from glucose, lactose, starch, cacao butter, hardened vegetable oil, kaolin, talc and other excipients; gum arabic powder, traganth powder, gelatin, ethanol and other binders; and laminaran, agar and other disintegrators and the like.

In some embodiments the pharmaceutical compositions of the present disclosure are in the form of capsules. Capsules are prepared according to ordinary methods by mixing carbostyril derivatives such as anhydrous aripiprazole crystals as the first ingredient and serotonin reuptake inhibitor as the second ingredient, and the various carriers described above and packing them in hard gelatin capsules, soft capsules hydroxypropylmethyl cellulose capsules (HPMC capsules) and the like.

In some embodiments the pharmaceutical compositions of the present disclosure are in the form of suppositories, which may include one or more pharmaceutically acceptable carriers or excipients selected from polyethylene glycol, cacao butter, higher alcohol. esters of higher alcohol, gelatin semi-synthetic glyceride and the like.

### Routes of Administration and Dosage Forms

Administration to a subject of the formulations according to the present disclosure may be via any common route so long as the target tissue is available via that route. The formulations may conveniently be presented in dosage unit form and may be prepared by any of the methods well known in the art of pharmacy. In some embodiments, the formulations are prepared by uniformly and intimately bringing the active components into association with a liquid carrier or a finely divided solid carrier or both, and then, if necessary, shaping the product into the desired dosage form. Of course, the skilled artisan will recognize that the active components (e.g. 2-Bromo-LSD) are included in an amount sufficient to produce the desired pharmacologic effect.

In some embodiments, the composition is administered depending on the type of preparation form, and the age, gender and other condition of the patient (degree and conditions of the disease, etc.). For example, tablets, pills, liquids, suspensions, emulsions, granules and capsules are administered orally. In case of an injectable preparation, it is administered intravenously by either singly or mixed with a common auxiliary liquid such as solutions of glucose or amino acid. Further, if necessary, the injectable preparation is singly administered intracutaneously, subcutaneously or intraperitoneally. In case of a suppository, it is administered intrarectally.

In some embodiments, 2-Bromo-LSD or a derivative or a salt thereof is administered at a dosage, such as described herein, at least once a day. In some embodiments, 2-Bromo-LSD or a derivative or a salt thereof is administered at a dosage, such as described herein, at least twice a day. In some embodiments, 2-Bromo-LSD or a derivative or a salt thereof is administered at a dosage, such as described herein, at least three times a day.

In other embodiments, 2-Bromo-LSD or a derivative or a salt thereof is administered at a dosage, such as described herein, at least once every other day. In yet other embodiments, 2-Bromo-LSD or a derivative or a salt thereof is administered at a dosage, such as described herein, at least once every third day. In further embodiments, 2-Bromo-LSD or a derivative or a salt thereof is administered at a dosage, such as described herein, at least once every fourth day. In further embodiments, 2-Bromo-LSD or a derivative or a salt thereof is administered at a dosage, such as described herein, or at least once every fifth day.

In some embodiments, the methods and formulations can be practiced as a single, one time dose or chronically. By chronic it is meant that the methods and compositions of the disclosure are practiced more than once to a given subject or individual. For example, chronic administration can be multiple doses of a pharmaceutical composition administered to a subject, on a daily basis, a weekly basis, a biweekly basis, monthly basis, or more or less frequently, as will be apparent to those of skill in the art. Chronic administration can continue for weeks, months, or years if appropriate according to the judgment of the practitioner of skill in the art. Furthermore, if certain doses, in the judgment of the practitioner of skill in the art, show tolerability profiles which may not be acceptable, the practitioner can reduce the dose to reduce such profiles.

### Aspects of the Invention

1. A method of treating major depressive disorder comprising or consisting of administering to a subject an amount of 2-Bromo-LSD or a derivative or salt thereof.
2. The method of claim 1, wherein the amount is a therapeutically effective amount of the 2-Bromo-LSD for administration at least once per day.
3. The method of claim 2, wherein the therapeutically effective amount ranges from about 27 μg to about 33 μg.
4. The method of claim 2, wherein the therapeutically effective amount is about 30 μg.
5. A method of treating major depressive disorder comprising or consisting of administering to a subject an amount of a pharmaceutical composition comprising 2-Bromo-LSD or a derivative or salt thereof and a pharmaceutically acceptable carrier or excipient.
6. The method of claim 5, wherein the pharmaceutical composition comprises about 27 μg to about 33 μg of 2-Bromo-LSD.
7. The method of claim 5, wherein the pharmaceutical composition comprises about 30 μg of 2-Bromo-LSD.
8. The method of claim S, wherein the amount is a therapeutically effective amount of the pharmaceutical composition for administration at least once per day.
9. The method of claim 5, wherein the pharmaceutical composition comprises a tablet or capsule.
10. The method of claim 5, wherein the pharmaceutical composition comprises an oral dosing solution.
11. A method of treating a mental disorder and/or a mood disorder comprising or consisting of administering to a subject an amount of 2-Bromo-LSD or a derivative or salt thereof, wherein the mental disorder and/or the mood disorder is Attention Deficit Hyperactivity Disorder, Obsessive-Compulsive Disorder, Atypical Depression. Melancholid Depression, Psychotic major depression, Catatonic Depression, Postpartum Depression, Premenstrual dysphoric disorder, Seasonal affective disorder, Dysthymia Double Depression, Depressive Disorder Not Otherwise Specified, Depressive personality disorder, Recurrent brief depression, and Minor Depressive disorder.
12. The method of claim 11, wherein the amount is a therapeutically effective amount of the 2-Bromo-LSD for administration at least once per day.
13. The method of claim 12, wherein the therapeutically effective amount ranges from about 10 μg to about 35 μg.
14. The method of claim 12, wherein the therapeutically effective amount ranges from about 15 μg to about 30 μg.
15. A method of treating major depressive disorder comprising or consisting of administering to a subject an amount of a pharmaceutical composition comprising 2-Bromo-LSD or a derivative or salt thereof and a pharmaceutically acceptable carrier or excipient, wherein the mental disorder and/or the mood disorder is Attention Deficit Hyperactivity Disorder, Obsessive-Compulsive Disorder, Atypical Depression, Melancholid Depression, Psychotic major depression, Catatonic Depression, Postpartum Depression, Premenstrual dysphoric disorder, Seasonal affective disorder, Dysthymia Double Depression, Depressive Disorder Not Otherwise Specified, Depressive personality disorder, Recurrent brief depression, or Minor Depressive disorder.
16. The method of claim 15, wherein the pharmaceutical composition comprises about 10 μg to about 35 μg of 2-Bromo-LSD.
17. The method of claim 15, wherein the pharmaceutical composition comprises about 15 μg to about 30 μg of 2-Bromo-LSD.
18. The method of claim 15, wherein the amount is a therapeutically effective amount of the pharmaceutical composition for administration at least once per day.
19. The method of claim 15, wherein the pharmaceutical composition comprises a tablet or capsule.
20. The method of claim 15, wherein the pharmaceutical composition comprises an oral dosing solution.
21. A method of treating a mental disorder and/or a mood disorder comprising or consisting of administering to a subject an amount of 2-Bromo-LSD or a derivative or salt thereof, wherein the mental disorder and/or the mood disorder is a bipolar disorder.
22. The method of claim 21, wherein the amount is a therapeutically effective amount of the 2-Bromo-LSD for administration at least once per day.
23. The method of claim 22, wherein the therapeutically effective amount ranges from about 27 μg to about 33 μg.
24. The method of claim 22, wherein the therapeutically effective amount is about 30 μg.
25. The method of claim 21, wherein the bipolar disorder is bipolar I, bipolar II, cyclothymia, or bipolar disorder not otherwise specified.
26. A method of treating major depressive disorder comprising administering to a subject an amount of a pharmaceutical composition comprising 2-Bromo-LSD or a derivative or salt thereof and a pharmaceutically acceptable carrier or excipient, wherein the mental disorder and/or the mood disorder is a bipolar disorder.
27. The method of claim 26, wherein the pharmaceutical composition comprises about 27 μg to about 33 μg of 2-Bromo-LSD.
28. The method of claim 26, wherein the pharmaceutical composition comprises about 30 μg of 2-Bromo-LSD.
29. The method of claim 26, wherein the amount is a therapeutically effective amount of the pharmaceutical composition for administration at least once per day.
30. The method of claim 26, wherein the pharmaceutical composition comprises a tablet or capsule.
31. The method of claim 26, wherein the pharmaceutical composition comprises an oral dosing solution.
32. The method of claim 26, wherein the bipolar disorder is bipolar I, bipolar II, cyclothymia, or bipolar disorder not otherwise specified.
33. A method of treating a mental disorder and/or a mood disorder comprising or consisting of administering to a subject an amount of 2-Bromo-LSD or a derivative or salt thereof, wherein the mental disorder and/or the mood disorder is selected from the group consisting of impulse-control disorders, adjustment disorders, and personality disorders.
34. The method of claim 33, wherein the amount is a therapeutically effective amount of the 2-Bromo-LSD for administration at least once per day.
35. The method of claim 34, wherein the therapeutically effective amount ranges from about 25 μg to about 50 μg.
36. The method of claim 34, wherein the therapeutically effective amount ranges from about 30 μg to about 45 μg.

37. A method of treating major depressive disorder comprising or consisting of administering to a subject an amount of a pharmaceutical composition comprising 2-Bromo-LSD or a derivative or salt thereof and a pharmaceutically acceptable carrier or excipient, wherein the mental disorder and/or the mood disorder is an impulse-control disorder, adjustment disorder, or personality disorder.

38. The method of claim 37, wherein the pharmaceutical composition comprises about 25 μg to about 50 μg of 2-Bromo-LSD.
39. The method of claim 37, wherein the pharmaceutical composition comprises about 30 μg to about 45 μg of 2-Bromo-LSD.
40. The method of claim 37, wherein the amount is a therapeutically effective amount of the pharmaceutical composition for administration at least once per day.
41. The method of claim 37, wherein the pharmaceutical composition comprises a tablet or capsule.
42. The method of claim 37, wherein the pharmaceutical composition comprises an oral dosing solution.
43. The method of any one of claims 37 to 42, wherein the composition comprises about 2.0 to about 3.5 2-Bromo-LSD, about 296.5 to about 298 mg microcrystalline cellulose, and about 3 mg of silicone dioxide or magnesium stearate.
44. An oral dissolving tablet for treatment of a mental disorder and/or a mood disorder that is an impulse-control disorder, adjustment disorder, or personality disorder, the composition comprising or consisting of:

| Oral Dissolving Tablet | mg |
|---|---|
| D-Mannitol | about 1.0 to about 162.5 |
| Xylitol | about 1.0 to about 162.5 |
| Microcrystalline Cellulose | about 1.0 to about 162.5 |
| Crospovidone | about 1.0 to about 162.5 |
| Dibasic Calcium Phosphate Anhydrous | about 1.0 to about 162.5 |
| 2-Bromo-LSD (based on patient body | about 2.0 to about 3.5 |
| weight) | |
| Magnesium Stearate 0.5% by weight | about 1.25 |

45. An oral dissolving capsule for treatment of a mental disorder and/or a mood disorder that is an impulse-control disorder, adjustment disorder, or personality disorder, the capsule comprising or consisting of a composition comprising or consisting of:

| | mg |
|---|---|
| 2-Bromo-LSD (based on patient body | about 2.0 to about 3.5 |
| weight) | |
| Microcrystalline Cellulose | about 296.5 to about |
| | 298.0 |
| Lubricant, Silicon Dioxide or Magnesium | about 3 |
| Stearate | |

46. An oral dissolving tablet for treatment of a mental disorder and/or a mood disorder that is an impulse-control disorder, adjustment disorder, or personality disorder, the tablet having a range of hardness at 100+/−50 Newtons, the tablet comprising or consisting of a composition comprising or consisting of:

| Oral Dosing in a Tablet | mg |
|---|---|
| 2-Bromo-LSD (based on patient body | about 2.0 to about 3.5 |
| weight) | |
| Microcrystalline Cellulose | about 296.5 to about |
| | 298.0 |
| Lubricant, Silicon Dioxide or Magnesium | about 3 |
| Stearate | |

### EXAMPLES

### Example 1—Oral Dosing in a Capsule

| Oral Dosing in a Capsule | mg |
|---|---|
| 2-Bromo-LSD (based on patient body | about 2.0 to about 3.5 |
| weight) | |
| Microcrystalline Cellulose | about 296.5 to about |
| | 298.0 |
| Lubricant, Silicon Dioxide or Magnesium | about 3 |
| Stearate | |

### Example 2—Oral Dosing in a Tablet Tablets have a range of hardness at 100+/−50 Newtons

| Oral Dosing in a Tablet | mg |
|---|---|
| 2-Bromo-LSD (based on patient body | about 2.0 to about 3.5 |
| weight) | |
| Microcrystalline Cellulose | about 296.5 to about |
| | 298.0 |
| Lubricant, Silicon Dioxide or Magnesium | about 3 |
| Stearate | |

### Example 3—Oral Dissolving Table

Tablets were pressed on a rotary tablet press to a compression range of about 34 to about 40 Newtons (N). At this tablet hardness, all tablets had a disintegration time of fewer than about30 seconds.

| Oral Dissolving Tablet | mg | |
|---|---|---|
| D-Mannitol | about 1.0 to about 162.5 | Diluent & |
| | | Palatability |
| Xylitol | about 1.0 to about 162.5 | Sweetener |
| Microcrystalline Cellulose | about 1.0 to about 162.5 | Binder |
| Crospovidone | about 1.0 to about 162.5 | Disintegrant |
| Dibasic Calcium Phosphate | about 1.0 to about 162.5 | Functional |
| Anhydrous | | Filler |
| 2-Bromo-LSD (based on | about 2.0 to about 3.5 | Active |
| patient body weight) | | |
| Magnesium Stearate 0.5% | about 1.25 | Lubricant |
| by weight | | |

### Example 4—Oral Dosing in Solution

| Oral Dosing in solution | mg | ml |
|---|---|---|
| 2-Bromo-LSD (based on patient body | about 2.0 to | |
| weight) | about 3.5 | |
| Distilled Water | | about 120 |

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1-46. (canceled)

47. A method of treating major depressive disorder comprising or consisting of administering to a subject an amount of 2-Bromo-LSD or a derivative or salt thereof, at least once per day.

48. The method of claim 47, wherein the 2-Bromo-LSD or a derivative or salt thereof may resolve symptoms associated with the major depressive disorder in subjects who have failed prior therapy.

49. The method of claim 47, wherein the amount ranges from about 24 μg/kg body weight to about 36 μg/kg body weight.

50. The method of claim 47, wherein the 2-Bromo-LSD or a derivative or salt thereof is provided as a composition comprising a pharmaceutically acceptable carrier or excipient.

51. The method of claim 50, wherein the pharmaceutical composition comprises a tablet, capsule, or an oral dosing solution.

52. A method of treating a mental disorder and/or a mood disorder comprising or consisting of administering to a subject an amount of 2-Bromo-LSD or a derivative or salt thereof at least once per day, wherein the mental disorder and/or the mood disorder is Attention Deficit Hyperactivity Disorder, Obsessive-Compulsive Disorder, Atypical Depression, Melancholid Depression, Psychotic major depression, Catatonic Depression, Postpartum Depression, Premenstrual dysphoric disorder, Seasonal affective disorder, Dysthymia Double Depression, Depressive Disorder Not Otherwise Specified, Depressive personality disorder, Recurrent brief depression. or Minor Depressive disorder, optionally wherein the 2-Bromo-LSD or a derivative or salt thereof may resolve symptoms associated with the mental disorder and/or a mood disorder in a subject who has failed prior therapy.

53. The method of claim 52, wherein the 2-Bromo-LSD or a derivative or salt thereof is provided as a composition comprising a pharmaceutically acceptable carrier or excipient.

54. The method of claim 52, wherein the amount ranges from about 10 μg/kg body weight to about 54 μg/kg body weight.

55. The method of claim 53, wherein the pharmaceutical composition comprises a tablet, capsule, or an oral dosing solution.

56. A method of treating bipolar disorder comprising or consisting of administering to a subject an amount of 2-Bromo-LSD or a derivative or salt thereof at least once per day,
optionally wherein the 2-Bromo-LSD or a derivative or salt thereof may resolve symptoms associated with the bipolar disorder a subject who has failed prior therapy.

57. The method of claim 56, wherein the amount ranges from about 27 μg/kg body weight to about 33 μg/kg body weight.

58. The method of claim 56, wherein the bipolar disorder is bipolar I, bipolar II, cyclothymia, or bipolar disorder not otherwise specified.

59. The method of claim 56, wherein the 2-Bromo-LSD or a derivative or salt thereof is provided as a composition comprising a pharmaceutically acceptable carrier or excipient.

60. The method of claim 59, wherein the pharmaceutical composition comprises a tablet, capsule, or an oral dosing solution.

61. A method of treating a mental disorder and/or a mood disorder comprising or consisting of administering to a subject an amount of 2-Bromo-LSD or a derivative or salt thereof at least one per day, wherein the mental disorder and/or the mood disorder is selected from the group consisting of impulse-control disorders, adjustment disorders, and personality disorders,
optionally wherein the 2-Bromo-LSD or a derivative or salt thereof may resolve symptoms associated with the mental disorder and/or a mood disorder a subject who has failed prior therapy.

62. The method of claim 61, wherein the amount ranges from about 25 μg to about 50 μg.

63. The method of claim 61, wherein the 2-Bromo-LSD or a derivative or salt thereof is provided as a composition comprising a pharmaceutically acceptable carrier or excipient.

64. The method of claim 63, wherein the pharmaceutical composition comprises a tablet, capsule or an oral dosing solution.

65. An oral dissolving tablet, capsule or tablet for administration at least once per day for treatment of a subject with a mental disorder and/or a mood disorder that is an impulse-control disorder, adjustment disorder, or personality disorder, the composition comprising or consisting of: 2-Bromo-LSD or a derivative or salt thereof and pharmaceutically acceptable carriers selected from one or more of D-mannitol, xylitol, microcrystalline cellulose, crosovidone, dibasic calcium phosphate anhydrous, silicon dioxide, and magnesium stearate,
optionally wherein the subject has failed prior therapy.

66. The oral dissolving tablet, capsule or tablet for administration according to claim 65, wherein the tablet has a range of hardness at 100+/−50 Newtons.
